(19) United States
(12) Patent Application Publication
(10) Pub. No.: US 2025/0302485 A1
(43) Pub. Date: Oct. 2, 2025

(54) CONNECTION ARRANGEMENT FOR CUFF AND CUFF INFLATION SYSTEM

(71) Applicant: Smart Tools Plus, LLC, Strongsville, OH (US)

(72) Inventors: Trevor Ruggiero, Strongsville, OH (US); Jared Ruehr, Strongsville, OH (US); Kendrick Langenbach, Strongsville, OH (US); Nicholas Colosi, Strongsville, OH (US)

(21) Appl. No.: 19/097,744

(22) Filed: Apr. 1, 2025

Related U.S. Application Data
(60) Provisional application No. 63/572,464, filed on Apr. 1, 2024.

(51) Int. Cl. A61B 17/135; A61H 9/00

(52) U.S. Cl. CPC A61B 17/135; A61H 9/0092; A61H 2201/168; A61H 2209/00

(57) ABSTRACT

Air pump connectors, ports, and holders that reduce damage to the connection between the inflation device and cuff and/or reduce pressure leakage between the inflation device and cuff so as to better control the inflation of the cuff by the inflation device and/or to better maintain the inflation of the cuff. The modified port on the cuff can be used inhibit or prevent the opposite air bladder surface from covering the bottom of the port, thus preventing the port from sealing. The modified port on the cuff can be configured to fit with the air-ports on an inflation device to inhibit or prevent the port on the cuff from dislodging from the housing of the inflation device, which dislodgement can result in air leakage between the inflation device and cuff.

### REFERENCED APPLICATIONS

The present application claims priority on U.S. Provisional Application Ser. No. 63/572,464 filed Apr. 1, 2024, all of which is incorporated herein by reference.

### FIELD OF DISCLOSURE

The present disclosure relates generally to blood flow restriction systems, specifically to a blood flow restriction system that can inflate a cuff to a limb occlusion pressure (LOP) or some upper operation pressure value (UOPV) when inflating the cuff positioned on a limb of a person or animal, particularly to novel air pump connectors, ports, and holders that reduce damage to the connection between the inflation device and cuff and/or reduce pressure leakage between the inflation device and cuff so as to between control and maintain the inflation of the cuff, and more particularly to a modified port on a cuff for use with a inflation device that inhibits or prevents the opposite air bladder surface from covering the bottom of the port, thus preventing the port from sealing, and/or inhibits or prevents the port on the cuff from dislodging from the air-port on the housing of the inflation device, which dislodgement can result in air leakage between the inflation device and cuff.

### BACKGROUND OF DISCLOSURE

Blood Flow Restriction Training (BFRT) has become a popular tool to improve muscle strength, size, and functional aerobic capacity in shorter amounts of time with less stress on the body than typical training. Practitioners began using BFRT in the treatment and recovery from musculoskeletal injuries or disabilities. Trainers and coaches have used BFRT as an adjunct for a usual training regimen or as a tool to aid recovery. BFRT is the brief and intermittent occlusion or restriction of arterial and venous blood flow performed by applying a tourniquet to the upper or lower extremity. BFRT has been found to augment skeletal muscle adaptation, along with systemic whole body changes and cardiovascular benefits while at rest, with low intensity endurance exercises or low load resistance training. BFRT has been found to be safe when applied with pressures relative to the cuff width and individual limb circumference which is obtained through measuring limb occlusion pressures via a Doppler.

BFRT is done by wrapping a tourniquet or cuff around the top portion of one's upper or lower limbs. The wrapping of the tourniquet or cuff, when inflated, restricts blood flow from the blood vessels of the working muscles to the heart and limits the amount of blood flow to the limbs from the blood vessels. The restriction results in physiological changes that mimic changes associated with high intensity exercise. The results are gains in muscle size and strength and increases in cardiovascular function at much lower intensities than are usually required for adaptation.

Various types of BFRT cuffs have been developed as described in U.S. Pat. Nos. 6,149,618; 7,413,527; 7,455,630; 8,021,283; 8,182,403; 8,273,114; 8,328,693; 8,366,740; 8,425,426; 8,992,397; 9,301,701; and 10,245,458; US Publication Nos. 2009/0124912; 2015/0150560; 2016/0193491; 2017/0112504; 2017/0224357; 2017/0325825; and 2018/0290005; PCT Publication Nos. WO 2016/087123; WO 2017/149690; and WO 2019/068147; and the Smart Cuff™ devices offered by Smart Tools Plus, LLC (https://www.smarttoolsplus.com/), all of which are fully incorporated herein by reference.

Manual and electric pumps are commonly utilized to inflate the cuff. Several prior art pump systems for inflating a cuff are disclosed in U.S. Pat. Nos. 10,646,231; 10,646,232; US Publication Nos. 2010/0324429; 2013/021126933; 2013/0211269; 2013/0184745; and 2021/0228099, all of which are fully incorporated herein by reference.

Portions of air bladders in prior art electronic pump control systems, cuff inflation systems, and BFRT systems are susceptible to isolation from the pump and air pressure sensors when the cuff is tightly wrapped around a limb. Because conventional cuff inflation systems have air bladders pressed directly onto the air-port holes, a seal is created that the pump is not strong enough to overcome. This is because when the air-port holes are blocked by the bladder, there is only a small surface area that pressure from the pump can engage to inflate the bladder. Thus, the small surface area and low air pressure provides only a small force to inflate the bladder/cuff. When air from the pump cannot freely move into the bladders/cuff, pressure in the cuff and blood flow pulsations cannot be accurately read and/or detected. Further, there is also risk of damaging the pump and/or bladder if the pump pressure cannot be accurately determined, for the pump may reach pressures beyond its specified maximum pressure.

Additionally, prior art electronic pump control systems, cuff inflation systems, and BFRT systems are easily susceptible to air leaks at the connection points between the air-ports and the pump during cuff/bladder inflation. For example, plastic inserts in the cuff that receive one or more air ports and/or sensor ports on the inflation device can be caused to slide during BFR use and/or the connection and/or disconnection of the inflation device from the cuff, thereby causing the air-ports to dislodge from connection with the inflation device.

In view of the current state of the art of inflation systems for cuffs, there is a need for (i) a modified port that inhibits or prevents the opposite air bladder surface from covering the bottom of the port, thus preventing the port from sealing; and (ii) a modified cuff port arrangement that fits with the air-port of a inflation device to inhibit or prevent the air-port from dislodging from the cuff.

### SUMMARY OF DISCLOSURE

The present disclosure relates to air pump connectors, ports, and holders that reduce damage to the connection between the inflation device and cuff and/or reduce pressure leakage between the inflation device and cuff so as to better control the inflation of the cuff by the inflation device and/or to better maintain the inflation of the cuff. In one non-limiting embodiment, there is provided a modified port on a cuff for use with an inflation device that inhibits or prevents the opposite air bladder surface from covering the bottom of the port, thus preventing the port from sealing. In another non-limiting embodiment, there is provided a modified port on a cuff that fits with the air-port on an inflation device to inhibit or prevent the air-port on the cuff from dislodging from the port on the housing of the inflation device, which dislodgement can result in air leakage between the inflation device and cuff.

In one non-limiting aspect of the present disclosure, there is provided a cuff that includes an inflation device, and wherein the cuff is formed one or more two layers of material. In one non-limiting embodiment, the cuff includes two layers of material coupled together to create one or more air chambers that can be inflated with air and/or some other gas or fluid. In another non-limiting configuration, the cuff is a single air chamber inflatable cuff. The configuration and shape of the one or more air chambers is non-limiting.

In another and/or alternative non-limiting aspect of the present disclosure, there is provided an inflation device (e.g., pump system, etc.) that can optionally be permanently or releasably secured to the cuff. In one non-limiting embodiment, the inflation device includes a housing that partially or fully includes and/or houses one or more of a) one or more pressure/pulse sensors, b) an air delivery unit, c) one or more circuit boards and other electronics, d) one or more pressure gauges, e) software, f) one or more displays (e.g., LED screen, OLED screen, etc.), g) GPS system, h) gyroscope, i) clock, j) timer, k) one or more processors (e.g., controller, etc.), l) wireless electronics (e.g., transmitter and/or receiver, etc.), m) air tubing, n) cuff connector, o) one or more lights (e.g., power light, Bluetooth or wireless connection light, power level light, charging indicator light, charging status light, update status light, etc.), and wherein one or more lights can perform one or more of the listed functions, p) one or more press or contact buttons (e.g., power button, pressure release button, home button, reset button, etc.), q) power/data transfer connector (e.g., DC barrel connectors, USB ports (including Type-A, Type-C, and Mini/Micro-B), AC power connectors, and specialized connectors (e.g., Molex, SATA, and PCIe power connectors), etc.), r) motor (e.g., electric motor, etc.), s) connection arrangement to permanently or releasably connect the housing to the cuff; t) one or more air ports; u) one or more valves, v) one or more temperature sensors, w) power source (e.g., rechargeable or non-rechargeable battery, etc.), and/or x) one or more pumps. In another non-limiting embodiment, the housing can be configured to be connected to an outer surface of the cuff. In another non-limiting embodiment, the housing has a maximum width that is the same or less than the width of the cuff at the location that the housing is connected to the cuff; however, this is not required. In another non-limiting embodiment, the housing has a length that is at least 0.5% (e.g., 0.5-30% and all values and ranges therebetween) the longitudinal length of the cuff; however, this is not required. In another non-limiting embodiment, the housing has a thickness of no more than 3 inches (e.g., 0.2-3 includes and all values and ranges therebetween); however, this is not required. In another non-limiting embodiment, the housing is configured to be maintained on the cuff during use of the inflated cuff by the user; this is not required. In another non-limiting embodiment, the inflation device can be used with the systems, apparatuses, and methods described in U.S. Patent Publication No. US 2024/0050100 (which is fully incorporated herein by reference) or the like to (i) provide a modified port assembly that inhibits or prevents one or more air bladders in cuff form being isolated from the inflation system when the cuff is tightly wrapped around a limb; and/or (ii) provide an port holder or connector that inhibits or prevents air leaks between the air delivery unit and cuff during the inflation of the cuff and/or use of the inflated cuff by a user.

In another and/or alternative non-limiting aspect of the present disclosure, there is provided a connection arrangement for an inflation device with the cuff that includes a housing having air-ports, a modified cuff port arrangement, a modified port cavity and a modified port insert. In one non-limiting embodiment, the modified port cavity can be optionally welded and/or adhesively connected to the air bladder that is located inside of cuff so as to create an air tight connection between the air bladder and the modified port cavity. The shape of the modified port cavity is non-limiting. In one non-limiting configuration, the shape of the modified port cavity is similar to the bottom edge portion of the connection arrangement on housing so as to form a snug fit between the modified port cavity and the connection arrangement when the housing is connected to the cuff. Such snug fit facilitates in reducing movement between the housing and cuff after connection of the housing to the cuff thereby a) reducing or prevent damage to the air-ports during use of the cuff by a user, and/or b) reducing or prevent air leakage between the air-ports and modified port cavity. In another non-limiting configuration, the modified cuff port arrange is welded and/or adhesively connected to bladder such that one or more ribs form a space within the bladder of the cuff at some predetermined distance. The shape, number and/or size of the ribs is non-limiting. In another non-limiting configuration, one or more bosses (e.g., cylindrical-shaped bosses, etc.) can be located on the modified port cavity, and can optionally be configured to extend through and out of the modified cuff cavity. The one or more bosses can include an opening that is configured to receive at least portion of the bottom portion of air-ports (e.g., telescopically receive, etc.) when the housing is connected to the cuff to form a connection (e.g., an airtight radial seal, etc.) between the one or more bosses and one or more air-ports. Sealing rings can optionally be used to facilitate in forming an air tight seal between one or more bosses and one or more air-ports. In another non-limiting configuration, the one or more bosses can optionally be a part of the modified cuff port arrangement, or the modified cuff port arrangement can be configured to engage one or more bosses that are extending from the modified port cavity. In another non-limiting configuration, the modified port cavity can be configured to form a tight fit between the modified port cavity and the connection arrangement when the housing is connected to the cuff so as to minimize or prevent movement of the housing relative to the cuff when the housing is connected to the cuff, wherein such minimizing of the movement of the housing relative to the cuff 104 reduces the risk of a) the one or more bosses and/or the one or more air-ports from breaking or being damaged during inflation and/or deflation of the cuff, b) the one or more bosses and/or the one or more air-ports from breaking or being damaged during the insertion or removal of the housing from the cuff, and/or c) the housing pulling or pushing the one or more bosses into the cuff, thus reducing or preventing interference with the inflation of the cuff by the air delivery unit.

In another and/or alternative non-limiting aspect of the present disclosure, there is provided a connection arrangement for an inflation device with the cuff that includes a modified cuff port arrangement that optionally includes one or more protrusion rings that are configured to be positioned about one or more bosses. The protrusion rings, when used, can be used to a) protect the one or more bosses from damage, b) facilitate in reducing or preventing air leakage between the one or more bosses and one or more air-ports when the housing is connected to the cuff, and/or c) facilitate in reducing movement between the housing and cuff when the housing is connected to the cuff.

In another and/or alternative non-limiting aspect of the present disclosure, there is provided a connection arrangement for an inflation device with the cuff wherein the modified port cavity in the cuff is configured to receive a portion of the housing of the inflation device. In one non-limiting configuration, the modified port cavity is configured to receive a portion of connection arrangement on the bottom of housing. The location of modified port cavity on cuff is non-limiting. In another non-limiting embodiment, the modified port cavity optionally includes one or more clips or other connection arrangements that are configured to releasable secure the housing to the modified port cavity.

In another and/or alternative non-limiting aspect of the present disclosure, when the one or more bosses are part of modified cuff port arrangement, one or more optional protrusion rings that are positioned on the top surface of modified cuff port arrangement are configured to encircle each of the bosses. When the one or more bosses are not part of modified cuff port arrangement, the one or more optional protrusion rings can be positioned on the top surface of modified cuff port arrangement and include openings that are configured to receive a portion of one or more bosses when the modified cuff port arrangement is inserted in modified port cavity. The shape of the protrusion rings is non-limiting (e.g., cylindrical shape, etc.). The protrusion rings, when used, can facilitate as forming a seal between the cuff and housing. The protrusion rings, when used, can be formed of an elastomeric material that can flex or compress so as to facilitate in forming an air-tight seal between the cuff and housing; however, other materials can be used.

In another and/or alternative non-limiting aspect of the present disclosure, when one or more ribs are used on the bottom surface of the modified cuff port arrangement, the one or more ribs can be configured to hold and/or form a space above and/or within the bladder of the cuff at some predetermined distance. The one or more ribs can be partially or fully positioned within the bladder to form spacing between the interior wall surfaces of the bladder. Alternatively or additionally, the one or more ribs, when used, can be positioned on the outer surface of the bladder and the gap formed by the ribs between the outer surface of the bladder and the bottom surface of modified cuff port arrangement enables the bladder to move upwardly and at least partially into such spacing formed by the ribs to allow for air flow into the bladder. When the cuff is wrapped around a limb, the gap or spacing formed by the one or more ribs a) that is created between the outer surface of the bladder and the bottom surface of modified cuff port arrangement allows for inflation to occur and/or to create a pathway for air to reach a less tightly wrapped part of the bladder where the cuff can more easily be inflated, b) that is created within the bladder by the one or more ribs that are located within the bladder allows for inflation to occur and/or to create a pathway for air so the cuff can more easily be inflated, and/or c) allows air from the air delivery unit to more freely enter the cuff, thereby allowing for more accurate limb occlusion pressure readings and more effective inflation of the cuff. In one non-limiting configuration, the air gap created by the one or more ribs located inside the bladder of the cuff is maintained (i.e., when the cuff is inflated and deflated).

In another and/or alternative non-limiting aspect of the present disclosure, the size of the modified cuff port arrangement is non-limiting. In one non-limiting configuration, the maximum width of the modified cuff port arrangement is at least 2 mm (e.g., 2-30 mm and all values and ranges therebetween). In another non-limiting configuration, the maximum length of the modified cuff port arrangement is at least 2 mm (e.g., 2-50 mm and all values and ranges therebetween). In another non-limiting configuration, the maximum height of the modified cuff port arrangement is at least 2 mm (e.g., 2-15 mm and all values and ranges therebetween. In another non-limiting configuration, the distance between the center-points of the protrusion rings is at least 1 mm (e.g., 1-50 mm and all values and ranges therebetween). In another non-limiting configuration, the maximum height of the cylindrical protrusion rings is at least 0.1 mm (e.g., 0.1-15 mm and all values and ranges therebetween. In another non-limiting configuration, the maximum height of the one or more ribs is at least 0.01 mm (e.g., 0.01-8 mm and all values and ranges therebetween). In another non-limiting configuration, the maximum width of the one or more ribs is at least 0.01 mm (e.g., 0.01-8 mm and all values and ranges therebetween). In another non-limiting configuration, the diameter of the openings in the one or more bosses is at least 0.1 mm (e.g., 0.1-6 mm and all values and ranges therebetween). The length or height of the bottom portion of the one or more bosses that extends below the bottom surface of modified cuff port arrangement is typically the same or greater than the height of the one or more ribs; however, this is not required. The length or height of the top portion of the one or more bosses that extends above the top surface of the body of the modified cuff port arrangement can be a) greater than or equal a thickness of the body, and/or b) the same or less than the height of protrusion ring.

In another and/or alternative non-limiting aspect of the present disclosure, the modified port insert can optionally be configured to prevent the end of the air ports on or extending from the housing of the inflation device when the housing is connected to the cuff from a) contacting the top surface of the cuff or bladder, b) penetrating into the cuff or bladder, or c) passing through one side of the bladder and contacting the interior of an the other side of the interior of the bladder. In one non-limiting embodiment, when the housing is connected to the cuff, the end of the air ports on or extending from the housing of the inflation device are configured to not extend fully through the modified port insert. In one non-limiting configuration, the modified port insert includes a landing that is configured to function as a stop to the ends of the air ports so as to precent the air ports from fully passing through the modified port insert when the housing is connected to the cuff. When the housing is connected to the cuff, the end of air ports either engage the landing or are spaced slightly above landing. Such a modified port insert configuration facilitates in a) inhibiting or preventing damage to the ends of air ports during the connection of the housing to the cuff, b) inhibiting or preventing damage to the bladder from the ends of air ports during the connection of the housing to the cuff, c) inhibiting or preventing damage to the ends of air-ports from contacting the bladder during the connection of the housing to the cuff, thus inhibiting or preventing compression of the bladder at the location of the bosses, which compression of the bladder can interfere with the initial inflation of the bladder and/or the obtaining of correct pressure reading from the bladder during the inflation process.

One non-limiting object of the present disclosure is the provision of an inflation device and inflatable cuff that is configured to reduce damage to the connection between the inflation device and cuff and/or reduce pressure leakage between the inflation device and cuff so as to better control the inflation of the cuff by the inflation device and/or to better maintain the inflation of the cuff.

In another and/or alternative non-limiting object of the present disclosure is the provision of an inflation device and inflatable cuff that includes a modified port on the cuff for use with an inflation device that inhibits or prevents the opposite air bladder interior surface from covering the bottom of the air-port when the inflation system is connected to the inflatable cuff, thus preventing the air-port from being partially or fully sealed against an interior surface of the cuff.

In another and/or alternative non-limiting object of the present disclosure is the provision of an inflation device and inflatable cuff that includes a modified port on a cuff that is configured to engage one or more air-ports on an inflation device to inhibit or prevent the one or more air-ports on the cuff from dislodging from the port on the housing of the inflation device.

In another and/or alternative non-limiting object of the present disclosure is the provision of an inflation device and inflatable cuff that includes a connection arrangement for an inflation device with the cuff.

In another and/or alternative non-limiting object of the present disclosure is the provision of an inflation device and inflatable cuff that includes one or more ribs form a space within the bladder of the cuff at some predetermined distance.

In another and/or alternative non-limiting object of the present disclosure is the provision of an inflation device and inflatable cuff that includes one or more bosses located on the modified port cavity.

In another and/or alternative non-limiting object of the present disclosure is the provision of an inflation device and inflatable cuff that includes one or more protrusion rings that are configured to be positioned about one or more bosses, which protrusion rings, when used, can be used to a) protect the one or more bosses from damage, b) facilitate in reducing or preventing air leakage between the one or more bosses and one or more air-ports when the housing is connected to the cuff, and/or c) facilitate in reducing movement between the housing and cuff when the housing is connected to the cuff.

In another and/or alternative non-limiting object of the present disclosure is the provision of an inflation device and inflatable cuff that includes the modified port insert can optionally be configured to prevent the end of the air ports on or extending from the housing of the inflation device when the housing is connected to the cuff from a) contacting the top surface of the cuff or bladder, b) penetrating into the cuff or bladder, or c) passing through one side of the bladder and contacting the interior of an the other side of the interior of the bladder.

In another and/or alternative non-limiting object of the present disclosure is the provision of a cuff system comprising an inflatable cuff that is configured to be releasably connected to a limb of a user, an inflation device that is configured to inflate the inflation cuff, and a cuff connection arrangement that is configured to connect the inflation device to the inflation cuff; the inflatable cuff includes an inflatable bladder; the inflation device includes a housing, one or more air-ports; the cuff connection arrangement includes a modified port cavity and a modified cuff port arrangement; the modified port cavity is connected to the inflation cuff; the modified port cavity includes one or more connectors that are configured to connected to the housing; the modified cuff port arrangement is connected to the inflation cuff; the modified cuff port arrangement includes a body and one or more ribs, one or more bosses, and/or one or more protrusion rings; the one or more ribs are positioned on a top surface of the cuff and/or are located in an interior of the inflatable bladder; the one or more protrusion rings each include an ring opening that is adapted to receive at least a portion of one of the air-ports when the inflation device is connected to the cuff; the one or more bosses each form an opening through the body of the modified cuff port arrangement.

In another and/or alternative non-limiting object of the present disclosure is the provision of a cuff system wherein the inflation device includes two of the air-ports and the cuff connection arrangement includes two of the protrusion rings wherein the ring opening in each of the protrusion rings is configured to telescopically received at least a portion of one of the air-ports.

In another and/or alternative non-limiting object of the present disclosure is the provision of a cuff system wherein the one or more protrusion rings are connected to or formed on the modified cuff port arrangement; at least a portion of the one or more protrusion rings extends above a top surface of the cuff; the one or more ribs are connected to or formed on the modified cuff port arrangement; the one or more ribs are positioned in an interior of the inflatable bladder.

In another and/or alternative non-limiting object of the present disclosure is the provision of a cuff system wherein the one or more protrusion rings are connected to or formed on a top surface of the body of the modified cuff port arrangement; the one or more ribs are connected to or formed on a bottom surface of the body of the modified cuff port arrangement; the ring opening in each of the protrusion rings includes a passageway through the protrusion ring that is at least partially aligned with the opening formed by one of the bosses.

In another and/or alternative non-limiting object of the present disclosure is the provision of a cuff system wherein the modified cuff port arrangement is formed of a single piece of material.

In another and/or alternative non-limiting object of the present disclosure is the provision of a cuff system wherein one or more of the bosses a) terminates at a top surface and a bottom surface of the body, b) extends upwardly from the top surface of the body and into at least a portion of an opening in the protrusion ring that is partially or fully aligned with the boss, and terminates at the bottom surface of the body, c) extends upwardly from the top surface of the body and into at least a portion of an opening in the protrusion ring that is partially or fully aligned with the boss, and extends downwardly from the bottom surface of the body, or d) terminates at the top surface of the body and extends downwardly from the bottom surface of the body.

In another and/or alternative non-limiting object of the present disclosure is the provision of a cuff system wherein one or more of the ribs is positioned adjacent to or closely adjacent to one or more of the bosses.

In another and/or alternative non-limiting object of the present disclosure is the provision of a cuff system wherein the modified port cavity is formed of a different material than the modified cuff port arrangement.

In another and/or alternative non-limiting object of the present disclosure is the provision of a cuff system wherein the one or more protrusion rings extend upwardly from a top surface of the modified port cavity.

In another and/or alternative non-limiting object of the present disclosure is the provision of a cuff system further including a modified port insert; the modified port insert is configured to be positioned above a top surface of the cuff and positioned at least partially about a portion of the one or more protrusion rings that extend upwardly from a top surface of the modified port cavity.

In another and/or alternative non-limiting object of the present disclosure is the provision of a cuff system wherein the modified port cavity includes a recessed cavity region; a bottom portion of the modified port insert is configured to be positioned in the recessed cavity portion.

In another and/or alternative non-limiting object of the present disclosure is the provision of a cuff system wherein the modified port cavity includes two port cavity openings; a portion of one of the protrusion rings is configured to be inserted through one of the port cavity openings.

In another and/or alternative non-limiting object of the present disclosure is the provision of a cuff system wherein each of the port cavity openings in the modified port cavity has a variable cross-sectional area along a longitudinal length of each of the port cavity openings; each of the port cavity openings in the modified port cavity includes a landing.

In another and/or alternative non-limiting object of the present disclosure is the provision of a cuff system wherein a bottom surface of the modified port cavity is spaced from a top surface of the cuff to form a gap region at or near the one or more protrusion rings.

In another and/or alternative non-limiting object of the present disclosure is the provision of an inflatable cuff that is adapted for use with an inflation device to inflate the inflatable cuff; the inflatable cuff includes an inflatable bladder and a cuff connection arrangement; the cuff connection arrangement is adapted to connect the inflation device to the cuff; the cuff connection arrangement includes a modified port cavity and a modified cuff port arrangement; the modified port cavity is connected to the inflation cuff; the modified port cavity includes one or more connectors that are configured to connected to the inflation device; the modified cuff port arrangement is connected to the inflation cuff; the modified cuff port arrangement includes a body and one or more ribs, one or more bosses, and/or one or more protrusion rings; the one or more ribs are positioned on a top surface of the cuff and/or are located in an interior of the inflatable bladder; the one or more protrusion rings each include an ring opening that is adapted to receive at least a portion of an air-port on the inflation device when the inflation device is connected to the cuff; the one or more bosses each form an opening through the body of the modified cuff port arrangement.

These and other advantages will become apparent to those skilled in the art upon the reading and following of this description.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1 illustrates an exemplary cuff system that can include a modified port and a modified cuff port arrangement in accordance with the present disclosure;

FIGS. 2A-C illustrates front, back, and side views of a non-limiting housing component of an inflation device that can be use with the cuff that is illustrated in FIG. 1;

FIG. 3 illustrates a non-limiting implementation of the modified port insert and the modified cuff port arrangement in accordance with the present disclosure;

FIG. 4 illustrates the modified port insert positioned about the modified port extending through a cuff insert in the cuff;

FIG. 5 depicts barbs/air-ports extending from the housing component of the inflation device that engages the modified port and the modified cuff port arrangement of FIG. 3;

FIGS. 6A-6D depict various views of the non-limiting modified port of FIG. 3, wherein FIG. 6A is a top view of the modified cuff port arrangement, wherein FIG. 6B is a bottom view of the modified cuff port arrangement, wherein FIG. 6C is a front view of the modified cuff port arrangement, and wherein FIG. 6D is a side view of the modified cuff port arrangement;

FIG. 7 is an exploded view of the exemplary cuff, housing component of the inflation device, and the modified port insert;

FIGS. 8A-8E depict various views of the modified port insert, wherein FIG. 8A is a top view of the modified port insert, wherein FIG. 8B is a bottom view of the modified port insert, wherein FIG. 8C is a cross-sectional, side view of the modified port insert, wherein FIG. 8D is a side view of the modified port insert, and wherein FIG. 8E is a perspective front view of the modified port insert.

### DESCRIPTION OF NON-LIMITING EMBODIMENTS

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

U.S. Patent Publication No. US 2024/0050100, entitled, “Method and Apparatus for Determining Limb Occlusion,” forms part of the disclosure of the present patent application, and the entire contents of such document are expressly fully incorporated by reference herein and is made part of this patent application for all purposes. The systems, apparatuses, and methods described in U.S. Patent Publication No. US 2024/0050100 relate to and facilitate the sensing of blood pressure in the limb of a human being (e.g., the upper arm, upper leg, etc.).

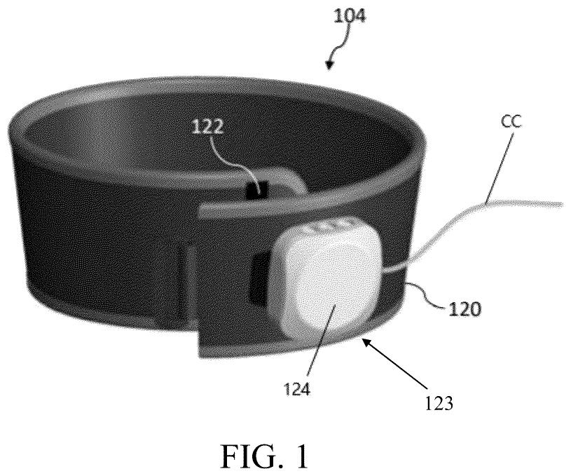

Referring now to FIG. 1, there is illustrated a non-limiting cuff 104 that includes an inflation device 123. The cuff 104 is formed one or more two layers of material. In one non-limiting embodiment, the cuff 104 includes two layers of material coupled together to create one or more air chambers that can be inflated with air and/or some other gas or fluid. In another non-limiting configuration, cuff 104 is a single air chamber inflatable cuff. In another non-limiting configuration, cuff 104 includes two or more air chambers. The configuration and shape of the one or more air chambers is non-limiting.

The inflation device 123 that is connected to cuff 104 is configured to inflate and/or deflate the cuff. The inflation device 123 can pressurize the cuff to produce compression on a target compression zone that in turn produces a partial or full restriction of blood flow in one or more blood vessels of a user. Cuff 104 can optionally include an outer material cover 120 having a cavity (not shown) for containing an inflatable bladder B to partially or fully isolate the inflatable bladder from the skin of the user and/or to provide protection to the inflatable bladder, and/or to provide structural strength to cuff 104.

The inflation device 123 can optionally be secured to the inner surface of outer material cover 120 (e.g., stitching, adhesive, melted connection, clip arrangement, snap arrangement, etc.). Outer material cover 120 can be generally made of a flexible and durable material (e.g., nylon, Kevlar™, etc.). In one non-limiting embodiment, outer material cover 120 is formed of an inelastic or non-stretch material. Outer material cover 120 can be configured to a) facilitate in distributing the force applied by the inflatable bladder B to the limb of a user, b) reduce pinching of the user's skin during the inflation of the cuff, and/or c) provide friction between cuff 104 and the user's skin to inhibit or prevent rotation of cuff 104 on the user during use.

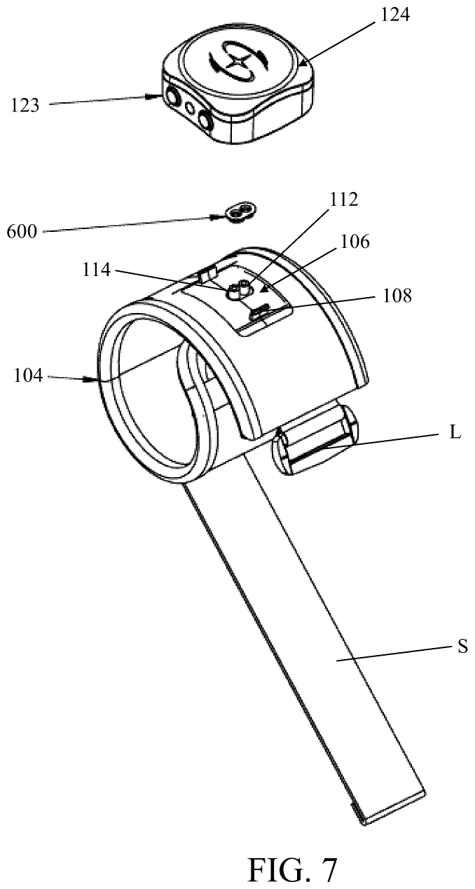

Cuff 104 generally includes a connection arrangement 122 to facilitate in the securing of cuff 104 to a user's limb. As illustrated in FIGS. 1 and 7, cuff 104 can include a “hook and loop” fastener 122 having a loop/loop-like material on the outer surface that is configured to releasably connect to a hook/loop material. A strap arrangement S that includes a “hook and loop” fastener L can also or alternatively be used to secure the cuff 104 to a user's limb. As can be appreciated, other types of connection arrangements can be used (e.g., buckles, snaps, etc.).

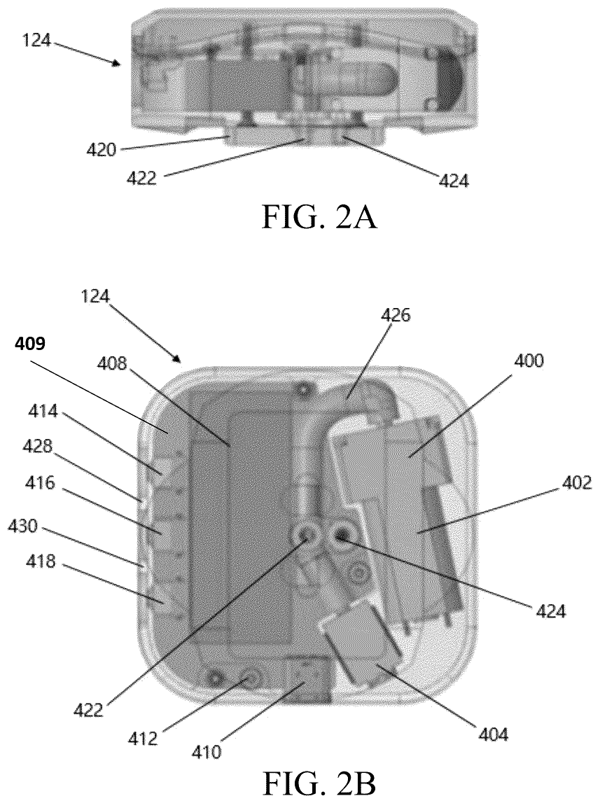

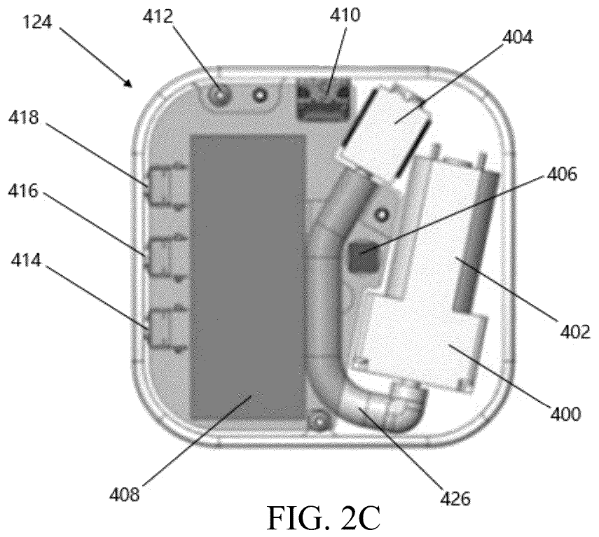

Continuing to refer to FIG. 1, cuff 104 can have an inflation device 123 mounted thereto. In one non-limiting configuration, inflation device 123 includes a housing 124 that partially or fully includes a pressure/pulse sensor and/or an air delivery unit. The housing 124 can be configured to be connected to an outer surface of outer material cover of the cuff 104. The cuff 104 can optionally include a housing connector to secure the housing 124 to the cuff 104. Generally, the housing 124 is configured to be releasably secured to the cuff; however, this is not required. Generally, the housing 124 is configured to be maintained on the cuff 104 during use of the cuff 104 by the user; however, the housing 124 can be configured to be removed from the cuff 104 during use of the cuff 104 by the user. As can be appreciated, housing 124 can be located on any portion of the outer material cover 120 of cuff 104. In one non-limiting embodiment, housing 124 partially or fully includes a pressure/pulse sensor and an air delivery unit, and when the housing 124 is connected to cuff 104, the housing 124 partially or fully overlies an air input port and/or a fluid sensor port on the cuff 104, and wherein the housing 124 and/or the air delivery unit and/or the pressure/pulse sensor include tubing and/or a connector that enables the air delivery unit to be fluidly connected to air input port and the pressure/pulse sensor to be fluidly connected to fluid sensor port when the housing is connected to the cuff 104. FIGS. 1, 2B and 2C illustrate an optional charging/data cable CC connected to the housing via a charging/data port 410.

Referring now to FIGS. 2A-2C, housing 124 of inflation device 123 is configured partially or fully includes the air delivery unit and the pressure/pulse sensor, and wherein the air delivery unit and the pressure/pulse sensor include, but not limited to, a) one or more motors; b) one or more pumps; c) optional power source; d) one or more pressure sensors and/or one or more pulse sensors; e) one or more circuit boards and other electronics; f) one or more processors (e.g., controller, etc.); g) optional one or more pressure gauges; h) software; i) optional one or more displays (e.g., LED screen, OLED screen, etc.); j) power/data transfer connector (e.g., DC barrel connectors, USB ports (including Type-A, Type-C, and Mini/Micro-B), AC power connectors, and specialized connectors (e.g., Molex, SATA, and PCIe power connectors), etc.); k) air delivery unit; l) optional GPS system; m) optional wireless electronics (e.g., transmitter and/or receiver, etc.) to send/receive wireless signals; n) optional one or more press or contact buttons (e.g., power button, home button, power off button, pressure release button, reset button, etc.); o) optional one or more selection or scroll buttons to enable manual operation/control of the air delivery unit; p) optional one or more switch(es) to enable manual operation/control of the air delivery unit; q) connection arrangement to permanently or releasably connect the housing to the cuff; r) optional one or more air ports; s) optional one or more valves, t) optional one or more air tubes, u) optional one or more temperature sensors, v) optional a gyroscope, w) optional one or more lights and/or other indicators to indicate a mode and/or operation and/or status of the air delivery unit and/or pressure/pulse sensor (e.g., power light, Bluetooth or wireless connection light, power level light, charging indicator light, charging status light, update status light, etc.); x) gyroscope; y) clock and/or z) timer. The shape and size of housing 124 is non-limiting. The material used to form the housing 124 is generally a durable material (e.g., plastic, metal, composite, etc.). In one non-limiting arrangement, the housing 124 is permanently or releasably connected to the cuff 104 and has a size and weight that does not interfere with the use of the cuff 104 while the housing 124 is connected to the cuff 104. Generally, the volume of the housing 124 of the air delivery unit 123 is no more than 500 cubic inches, typically 0.5-500 cubic inches (and all values and ranges therebetween), and more typically 1-50 cubic inches. Generally, the weight of the housing 104 and of the air delivery unit and contents therein, including housing, is no more than 10 lbs., typically 0.05-10 lbs. (and all values and ranges therebetween), and more typically 0.1-1 lbs.

Referring again to FIGS. 2A-2C, there is illustrate front, back, and side views of one non-limiting housing 124 of the air delivery unit 123 and non-limiting components that are partially or fully contained in the housing 124. As illustrated in FIGS. 2A-2C, the housing 124 can contain a) a motor/pump 400; b) a motor portion 402; c) a pressure release value 404; d) a pressure sensor/pulse sensors 406, e) a battery 408; f) a circuit board/processor 409; g) a power port/data port 410 (e.g., micro USB port, etc.); h) wireless electronics (e.g., transmitter and/or receiver, etc.) positioned near screw 412; i) buttons 428, 430 (e.g., power button, home button, power off button, reset button, etc.); k) connection arrangement 420 to permanently or releasably connect the housing to the cuff 104; l) air-ports 422, 424; m) air tubes 426, and n) one or more lights 414, 416, 418 and/or other indicators to indicate a mode and/or operation and/or status of the air delivery unit and/or pressure/pulse sensor.

As illustrated in FIGS. 2A-2C, a short air tube 426 (e.g. tube that is less than five inches, etc.) is positioned internally in the housing and forms a fluid connection between motor/pump 400 and air-port 422. Air tube 426 can optionally be connected to pressure release valve 404 that, when opened, depressurizes the cuff 104. Pressure sensor/pulse sensor 406 is illustrated as being directly connected to air-port 424. In the present non-limiting embodiment, a tube that is substantially less (e.g., 0.1-30% the length of air tube 426 and all values and ranges therebetween) than the length of air tube 426 is used to fluidly connect pressure sensor/pulse sensor 406 to air-port 424. In other non-limiting embodiments, no tube is used to connect pressure sensor/pulse sensor 406 to air-port 424. As illustrated in FIG. 2A, air-ports 422, 424 extend downwardly from an underside of housing 124. As also illustrated in FIG. 2A, the air-ports 422, 424 optionally do not extend downwardly beyond a bottom edge of connection arrangement 420.

The disclosed technology can be used with the systems, apparatuses, and methods described in U.S. Patent Publication No. US 2024/0050100 or the like to (i) provide a modified port assembly that inhibits or prevents one or more air bladders in the cuff from being isolated from the inflation system when the cuff is tightly wrapped around a limb; and/or (ii) provide an port holder or connector that inhibits or prevents air leaks between the air delivery unit and cuff during the inflation of the cuff and/or use of the inflated cuff by a user.

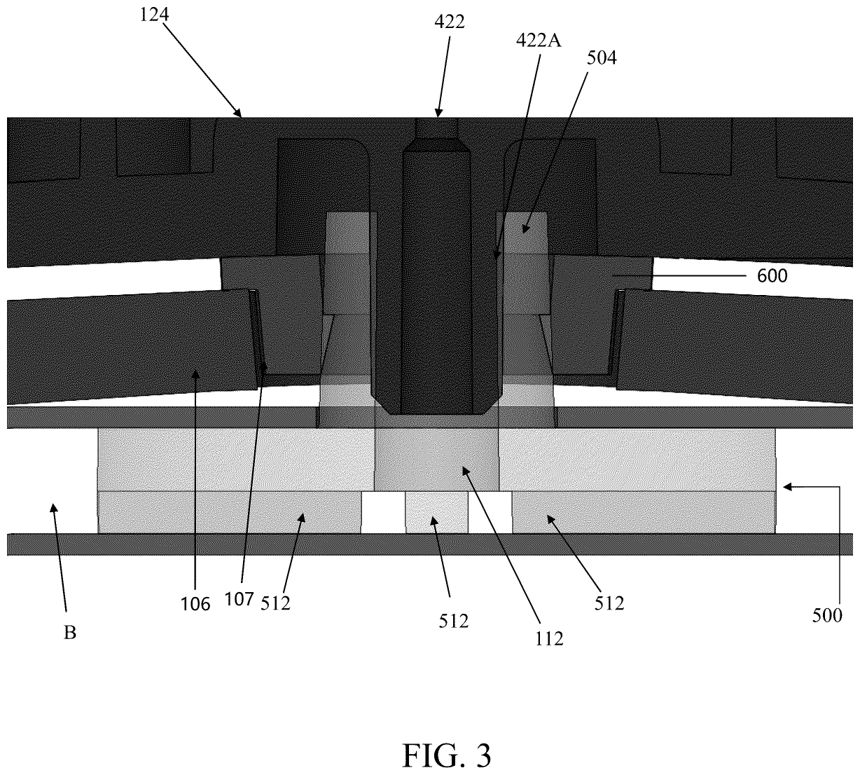

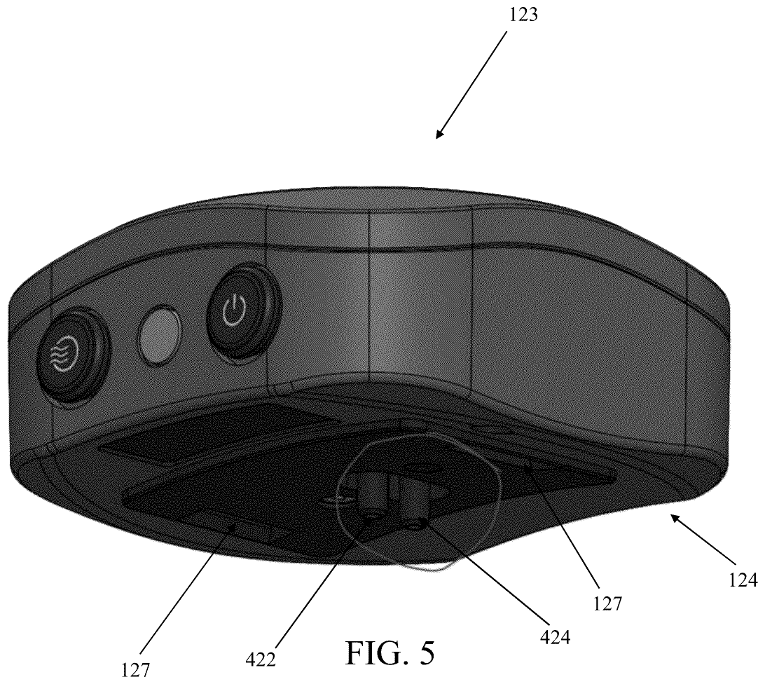

Referring now to FIG. 3, there is illustrated a non-limiting configuration of an enlarged portion of housing component 124 having air-ports 422, 424 (see also FIG. 5), an enlarged modified cuff port arrangement 500, an enlarged portion of modified port cavity 106 and an enlarged portion of modified port insert 600. In one non-limiting embodiment, modified port cavity 106 can be optionally welded or otherwise connected (e.g., adhesive connection, melted and/or heat bonded connection, clamp connection, etc.) to the air bladder B (e.g., polyurethane bladder, etc.) of cuff 104 to create an air tight connection between the air bladder B and the modified port cavity 106. All or a portion of the modified port cavity 106 can be positioned on the top surface of the air bladder B and/or positioned in the interior of the air bladder B.

The all or a portion of the shape of the top portion of the modified port cavity 106 can be similar in shape to the bottom edge portion of the connection arrangement 420 on housing 124 so as to form a snug fit between the modified port cavity 106 and the connection arrangement 420 when the housing is connected to the cuff; however, this is not required. Such snug fit, when established, can facilitate in reducing movement between the housing and cuff after connection of the housing to the cuff, thereby a) reducing or prevent damage to the air-ports 422, 424 during use of the cuff by a user, and/or b) reducing or prevent air leakage between the air-ports 422, 424 and modified port cavity 106. In another non-limiting embodiment, modified cuff port arrangement 500 is welded and/or otherwise connected (e.g., adhesive connection, melted and/or heat bonded connection, clamp connection, etc.) to bladder B such that ribs 512 form a space within the interior of the bladder B of the cuff at some predetermined distance. All of the modified cuff port arrangement 500 can be located within the interior of the bladder B, or a portion of the modified cuff port arrangement 500 can be located within the interior of the bladder B and a portion of the modified cuff port arrangement 500 can be located outside the interior of the bladder B.

Bosses 112, 114 (e.g., cylindrically-shaped bosses, etc.) can be configured to a) receive at least a bottom portion of air-ports 422, 424 when the housing is connected to the cuff, or b) be spaced from the bottom portion of air-ports 422, 424 when the housing is connected to the cuff. In one non-limiting configuration, bosses 112, 114 can be part of modified cuff port arrangement 500. The bosses 112, 114 can be a) merely an opening in the body 510 of the modified cuff port arrangement 500, and/or b) be or include an extension that extends upwardly from the top surface of the body 510 of the modified cuff port arrangement 500. In one non-limiting embodiment, the bosses 112, 114 are merely an opening in the body 510 of the modified cuff port arrangement 500. In one non-limiting embodiment, the bosses 112, 114 have an opening respectively that is configured to a) receive at least a bottom portion of air-ports 422, 424 (e.g., telescopically receive, etc.) when the housing is connected to the cuff to form an airtight radial seal between the bosses and the air-ports, and/or b) receive air from the bottom portion of air-ports 422, 424 during the inflation of the cuff. When a portion of the bosses 112, 114 are configured to receive a portion of the bottom portion 422A of air-ports 422, 424, the openings 516, 518 generally have the same or similar shape as the outer perimeter of the air-ports 422, 424, and have a size so as to form a tight fit between the inserted portion of the air-ports 422, 424 in the openings in the bosses 112, 114. Bosses 112, 114 can be a part of modified cuff port arrangement 500 (e.g., merely be openings in the modified cuff port arrangement 500), or alternatively the modified cuff port arrangement 500 can be configured to engage bosses 112, 114 that are extending from modified port cavity 106. As illustrated in FIG. 3, the bosses 112, 114 are part of modified cuff port arrangement 500.

As illustrated in FIG. 3, the bottom of the bosses 112, 114 is spaced from an interior surface of bladder B. Such spacing can be facilitated by one or more ribs 512. The spacing of the bottom of the bosses 112, 114 from an interior surface of bladder B inhibits or prevents air flow blockage or impairment during the inflation and/or deflation. The size of the spacing is non-limiting (e.g., at least 0.001 mm; 0.001-10 mm and all values and ranges therebetween).

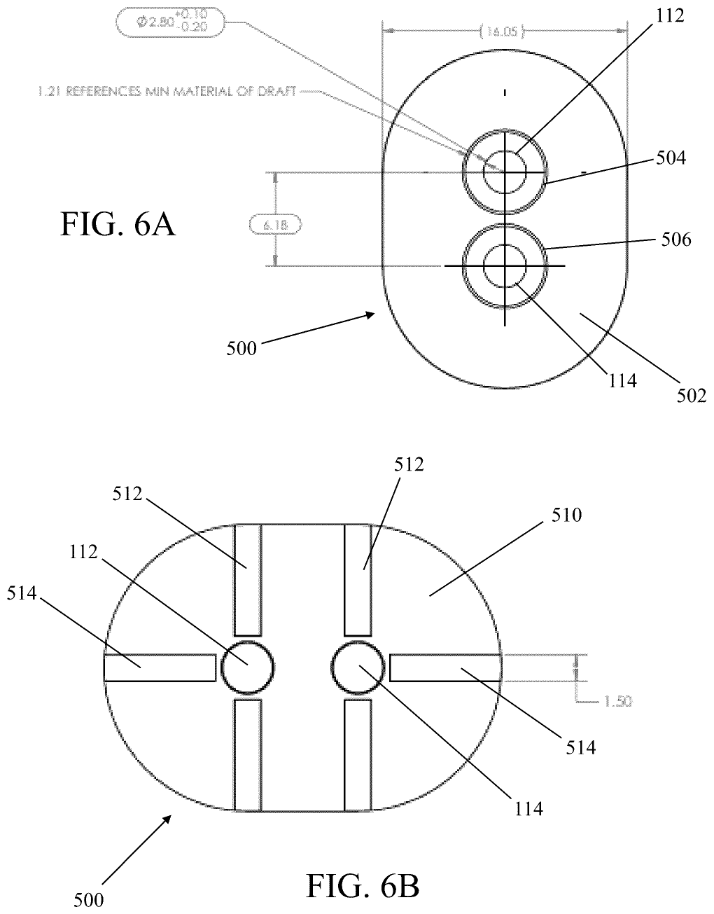

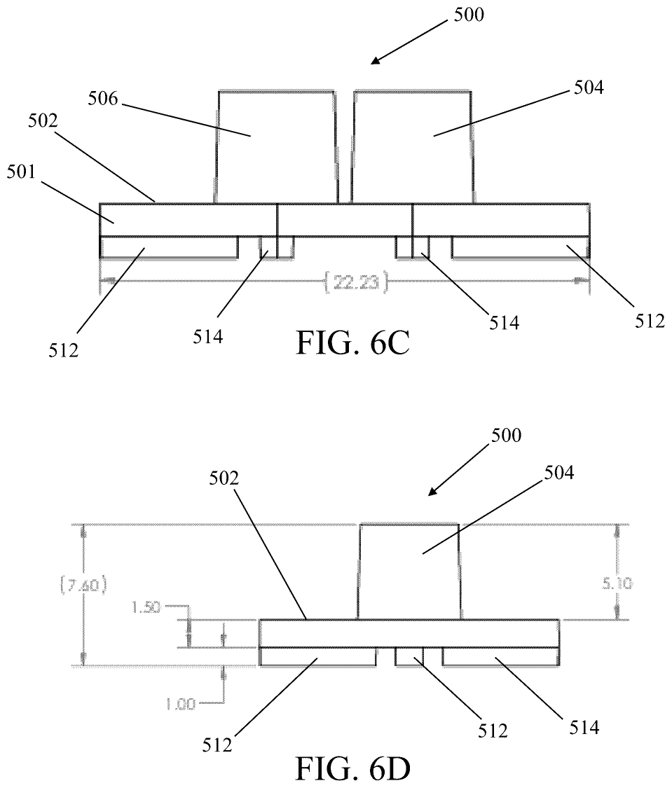

The one or more protrusion rings 504, 506 can be a) positioned above the top opening of the bosses 112, 114 (which bosses 112, 114 are merely an opening in the body 510 of the modified cuff port arrangement 500), or b) positioned about a portion or all of the bosses 112, 114 that extends upwardly from a top surface of the body of the modified cuff port arrangement 500. As illustrated in FIG. 6A, the protrusion rings 504, 506 are spaced apart from one another; however, this is not required. The protrusion rings 504, 506 can be used to a) protect the portion of bosses 112, 114 that extends upwardly from a top surface of the body of the modified cuff port arrangement 500 from damage, b) facilitate in reducing or preventing air leakage between the bladder B and air-ports 422, 424 when the housing is connected to the cuff, and/or c) facilitate in reducing movement between the housing and cuff when the housing is connected to the cuff. The protrusion rings 504, 506 can be formed of a hard material (e.g., hard plastic, metal, etc.) or a compressible material (e.g., compressible plastic material, foam material, etc.). The protrusion rings 504, 506 can a) optionally be connected to the outer surface of the bosses 112, 114 (e.g., adhesive connection, melted and/or heat bond connection, etc.), and/or be frictionally connected when a portion of the bosses 112, 114 extends upwardly from a top surface of the body of the modified cuff port arrangement 500, and/or b) connected to or formed on the body of the modified cuff port arrangement 500. In one non-limiting configuration, the bosses 112, 114 are connected to formed on the body of the modified cuff port arrangement 500. As can be appreciated, bosses 112, 114 can be connected to or be formed on modified port cavity 106.

The modified port cavity 106 can optionally be formed of a hard material (e.g., plastic material, metal material, ceramic material, etc.) that facilitates to help give the modified port cavity 106 of cuff 104 a generally fixed shape and to facilitate in creating a strong and secure mechanical connection between the cuff 104 and the housing 124. The modified port cavity 106 can be configured to form a tight fit between the modified port cavity 106 and the connection arrangement 420 when the housing is connected to the cuff 104 so as to minimize or prevent movement of the housing 124 relative to the cuff 104 when the housing is connected to the cuff 104. Such minimizing of the movement of the housing relative to the cuff 104 reduces the risk of a) protrusion rings 504, 506, bosses 112, 114 and/or air-ports 422, 424 from breaking or being damaged during inflation and/or deflation of the cuff, and/or b) protrusion rings 504, 506, bosses 112, 114 and/or air-port 422, 424 from breaking or being damaged during the insertion or removal of the housing from the cuff.

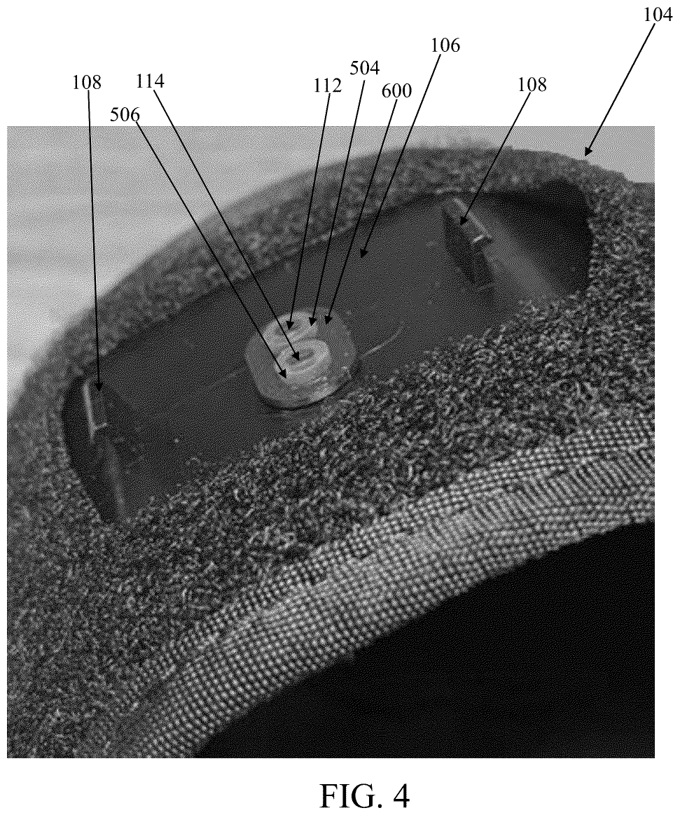

Referring now to FIGS. 3 & 4, there is illustrated the modified port cavity 106 in cuff 104 that is configured to receive a portion of housing 124. In one non-limiting configuration, the modified port cavity 106 is configured to receive a portion of connection arrangement 420 on the bottom of housing 124. The location of modified port cavity 106 on cuff 104 is non-limiting. Modified port cavity 106 optionally includes one or more clips 108 or other connection arrangements that are configured to releasably or non-releasably secure the housing 124 to the modified port cavity 106. As illustrated in FIG. 4, two clips 108 are spaced from one another and extend upwardly from the top surface of the modified port cavity 106 and include a top engagement flange used to be secured to housing 124. Two protrusion rings 504, 506 are illustrated as extending upwardly from the top surface of the modified port cavity 106. As discussed above, the protrusion rings 504, 506 can be part of the modified port cavity 106 or part of the modified cuff port arrangement 500. Also illustrated, the protrusion rings 504, 506 are positioned above the top openings of the bosses 112, 114.

A modified port insert 600 is illustrated as being inserted about the protrusion rings 504, 506. The modified port insert 600 is configured in combination with the protrusion rings 504, 506 to inhibit or prevent air-ports/barbs 422, 424 of housing 124 from dislodging or disengaging from the modified port cavity 104 as cuff insert 104 moves/slides during BFR use. As can be appreciated, optional sealing rings (not shown) can be used to facilitate in inhibiting or preventing air from escaping between protrusion rings 504, 506 and air-ports 422, 424 of housing 124. The modified port insert 600 is illustrated as including two openings to each receive one of the protrusion rings 504, 506; however, this is not required. The material used to for the modified port insert 600 is non-limiting (e.g., plastic, metal, ceramic, etc.). Generally, the material used to form the modified port insert 600 is different from the material used to form the protrusion rings 504, 506. The thickness of the modified port insert 600 is illustrated as being less than the height of the protrusion rings 504, 506 that extend upwardly from the top surface of the modified port insert 600 (e.g., extend 2-60% and all values and ranges therebetween of the height of the protrusion rings 504, 506 that extend upwardly from the top surface of the modified port insert 600). As illustrated in FIG. 3, the bottom surface of the modified port insert 600 can be configured to extend downwardly into a cavity region 107 of the modified port cavity 106. Such a configuration facilitates in maintaining the position of the modified port insert 600 relative to the modified port cavity 106. As illustrated in FIG. 3, the top portion of the modified port insert 600 has a cross-sectional area that is greater that the cross-sectional area of the portion of the modified port insert 600 that is located below the top portion. The portion of the modified port insert 600 that is located below the top portion can optionally be tapered. As illustrated in FIG. 3, cavity region does not fully pass through modified port cavity 106; however, this is not required. As illustrated in FIG. 3, the bottom of cavity region 107 includes one or more openings that are sized to enable one or both bosses 112, 114 and/or one or both protrusion rings 504, 506 to pass through the bottom of cavity region 107.

Referring now to FIG. 5, there is illustrated housing 124 that includes non-limiting air-ports 422, 424 in the bottom of housing 124. The air-ports 422, 424 are configured to engage the bosses 112, 114 and/or protrusion rings 504, 506. In one non-limiting configuration the exterior shape of the air-ports 422, 424 and the interior opening of the protrusion rings 504, 506 have a circular cross-sectional shape such that a portion of the air-ports 422, 424 is telescopically received in the interior opening of the protrusion rings 504, 506 when the housing 124 is connected to the cuff 104. As can be appreciated, the cross-sectional shape of the exterior shape of the air-ports 422, 424 and the interior opening of the protrusion rings 504, 506 can have other shapes (e.g., oval, polygonal, etc.). As illustrated in in FIG. 5, the bottom portion of the air-ports 422, 424 extends below the bottom surface of the housing 124; however, this is not required.

Again referring to FIG. 5, the bottom surface of the housing 124 includes two clip cavities 127 that are configured to receive a portion of clip 108 when the housing 124 is connected to the cuff 104. As also illustrated in FIG. 5, the side of the housing 124 includes two buttons and a single light 414 located between the two buttons 428. As can be appreciated, other button and light configurations can be used.

Referring now to FIGS. 6A-6D, there is depict various non-limiting views of the modified cuff port arrangement 500. FIG. 6A is a top view of modified cuff port arrangement 500. FIG. 6B is a bottom view of modified cuff port arrangement 500. FIG. 6C is a front view of modified cuff port arrangement 500. FIG. 6D is a side view of modified cuff port arrangement 500. The dimensions included on FIG. 6A is only representative of one non-limiting embodiment, and it will be appreciated that the components of the modified cuff port arrangement 500 can have other dimensions.

The modified cuff port arrangement 500 includes body 501 that has a top surface 502 and a bottom surface 510. In one non-limiting embodiment, protrusion rings 504, 506 extend upwardly from the top surface 502 of the modified cuff port arrangement 500. The protrusion rings 504, 506 are hollow and include openings 516, 518 to allow air flow thereto. The length of the openings 516, 518 can be the same as, longer than or shorter than the length of the air-ports 422, 424 that extend downwardly from an underside of housing 124. The protrusion rings 504, 506 can be formed part of modified cuff port arrangement 500 or be separate components of modified cuff port arrangement 500. When the protrusion rings 504, 506 are separate components of modified cuff port arrangement 500, modified cuff port arrangement is configured to be positioned about a portion of the protrusion rings 504, 506, the protrusion rings 504, 506 are coaxial with openings formed in the top surface 502 and bottom surface 510 of modified cuff port arrangement 500.

In one non-limiting configuration, the protrusion rings 504, 506 are part of modified cuff port arrangement 500, and the protrusion rings 504, 506 that are positioned on the top surface 502 of modified cuff port arrangement 500 a) encircle each of cylindrical bosses 112, 114 when the cylindrical bosses 112, 114 extend upwardly from the top surface of body 501, or b) the openings 516, 518 in the protrusion rings 504, 506 are partially of fully aligned with the opening in the bosses 112, 114 when the cylindrical bosses 112, 114 do not extend upwardly from the top surface of body 501. The protrusion rings 504, 506 are illustrated as having a generally cylindrical shape; however, other shapes can be used. The protrusion rings 504, 506 are used to facilitate in forming a seal between the cuff and housing. The protrusion rings 504, 506 can be formed of an elastomeric material that can flex or compress so as to facilitate in forming an air-tight seal between the cuff and housing. It is to be understood that protrusion rings 504, 506 are not limited to a cylindrical shape and can be any reasonable shape (rectangular, square, conical, etc.). Likewise, openings 516, 518 in the protrusion rings 504, 506 can correspond to the shape of bosses 112, 114 when the bosses extend upwardly from the top surface of the body 501 of the modified cuff port arrangement 500, and/or the shape of the outer surface of the air-ports 422, 424 that extend downwardly from an underside of housing 124.

Ribs 512, 514 are illustrated as being positioned on the bottom surface 510 of modified cuff port arrangement 500. In one non-limiting configuration, one or more ribs are located directly adjacent to closely adjacent to (e.g., 0.001-1 mm and all values and ranges therebetween) one or more bosses 112, 114. The ribs 512, 514 are configured to hold/maintain and/or form a space above and/or within the bladder B of the cuff at some predetermined distance, and generally hold/maintain and/or form a space above and/or within the bladder B of the cuff at a location that is directly adjacent to closely adjacent to (e.g., 0.001-1 mm and all values and ranges therebetween) one or more bosses 112, 114. The ribs 512, 514 can be partially or fully positioned within the bladder B to create spacing in the bladder B. Alternatively, the ribs can be positioned on the outer surface of the bladder B, and the gap formed by the ribs between the outer surface of the bladder B and the bottom surface 510 of modified cuff port arrangement 500 enables the bladder to move upwardly and at least partially into such spacing formed by the ribs to allow for air flow into the bladder. Thus, when the cuff 120 is tightly wrapped around a limb, the gap or spacing formed by the ribs 512, 514 that is a) created between the outer surface of the bladder and the bottom surface 510 of modified cuff port arrangement 500 allows for inflation to occur and/or to create a pathway for air to reach a less tightly wrapped part of the bladder where the cuff can more easily be inflated, or b) created within the bladder B by the ribs that are located within the bladder allows for inflation to occur and/or to create a pathway for air so the cuff can more easily be inflated. Furthermore, the configuration of the rib 512, 514 on the bottom surface 510 of modified cuff port arrangement 500 allows air from the air delivery unit 123 to more freely enter the cuff 120, thereby allowing for more accurate limb occlusion pressure readings and more effective inflation of the cuff 120. In one non-limiting embodiment, the air gap created in the bladder of cuff 120 by the modified cuff port arrangement 500 is maintained (i.e., when the cuff is inflated and deflated) by the ribs 512, 514. In another non-limiting embodiment, the ribs on the modified cuff port arrangement 500 are positioned on an outer surface of the bladder. In one non-limiting configuration, the bottom of the bosses 112, 114 terminates at the bottom of body 501 of the modified cuff port arrangement 500, and/or the bosses 112, 114 extends downwardly from the bottom of the body 501 of the modified cuff port arrangement 500 a distance that is less than the distance the ribs extends downwardly from the body 501 of the modified cuff port arrangement 500. In another non-limiting embodiment, the thickness of the ribs is less than or equal to the thickness in the body 501 of the modified cuff port arrangement 500. As illustrated in FIG. 6B, multiple ribs are located directly adjacent to closely adjacent to (e.g., 0.001-1 mm and all values and ranges therebetween) one or more bosses 112, 114.

With further reference to FIGS. 6A-6D, the modified cuff port arrangement 500 can optionally be formed from one or more thermoplastic polyurethane materials; however, other materials can be used. In one non-limiting embodiment, the width of the body 501 of the modified cuff port arrangement 500 is at least 2 mm, and typically 2-30 mm (and all values and ranges therebetween), and in one non-limiting configuration is 16.05 mm. In another non-limiting embodiment, the length of the body 501 of the modified cuff port arrangement 500 is at least 4 mm, and typically 4-50 mm (and all values and ranges therebetween), and in one non-limiting configuration is 22.23 mm. In another non-limiting embodiment, the height of the body 501 of the modified cuff port arrangement 500 is at least 2 mm, and typically 2-15 mm (and all values and ranges therebetween), and in one non-limiting configuration is 7.60 mm. In another non-limiting embodiment, the distance between the center-points of protrusion rings 504, 506 is at least 1 mm, and typically 1-20 mm (and all values and ranges therebetween), and in one non-limiting configuration is 6.18 mm. In another non-limiting embodiment, the height of the cylindrical protrusion rings 504, 506 is at least 1 mm, and typically 1-15 mm (and all values and ranges therebetween), and in one non-limiting configuration is 5.10 mm. In another non-limiting embodiment, the height of the ribs 512, 514 is at least 0.1 mm, and typically 0.1-8 mm (and all values and ranges therebetween), and in one non-limiting configuration is 1 mm. In another non-limiting embodiment, the width of the ribs 512, 514 is at least 0.2 mm, and typically 0.2-8 mm (and all values and ranges therebetween), and in one non-limiting configuration is 1.50 mm. The number of ribs 512, 514 on bottom surface 510 is non-limiting. The thickness of body 501 is generally greater than the height of the ribs 512, 514; however, this is not required. In another non-limiting embodiment, the diameter of the openings in protrusion rings 504, 506 is at least 0.5 mm, and typically 0.5-6 mm (and all values and ranges therebetween), and in one non-limiting configuration is 2.80 mm. In one non-limiting configuration, the modified cuff port arrangement 500 is formed of a single piece of material that includes the body, one or more protrusion rings 504, 506, one or more ribs 512, 514, and one or more bosses 112, 114 in the body wherein each boss is partially or fully aligned with the opening in each of the protrusion rings 504, 506. In another non-limiting configuration, the modified cuff port arrangement 500 is formed of multiple pieces of material (which multiple pieces can be formed of the same or different material), and wherein the modified cuff port arrangement 500 includes a body 501, one or more protrusion rings 504, 506 that are formed on or connected to the body (e.g., adhesive connection, melted and/or heat formed connection, etc.), one or more ribs 512, 514 that are formed on or connected to the body (e.g., adhesive connection, melted and/or heat formed connection, etc.), and one or more bosses 112, 114 in the body wherein each boss is partially or fully aligned with the opening in each of the protrusion rings 504, 506.

FIG. 7 depicts an exploded configuration of cuff 104, housing 124 of air delivery unit 123, and modified port insert 600. The cuff 104 is illustrated as including a strap S and a strap connector L that can be used to releasably connect the cuff to a limb of a user.

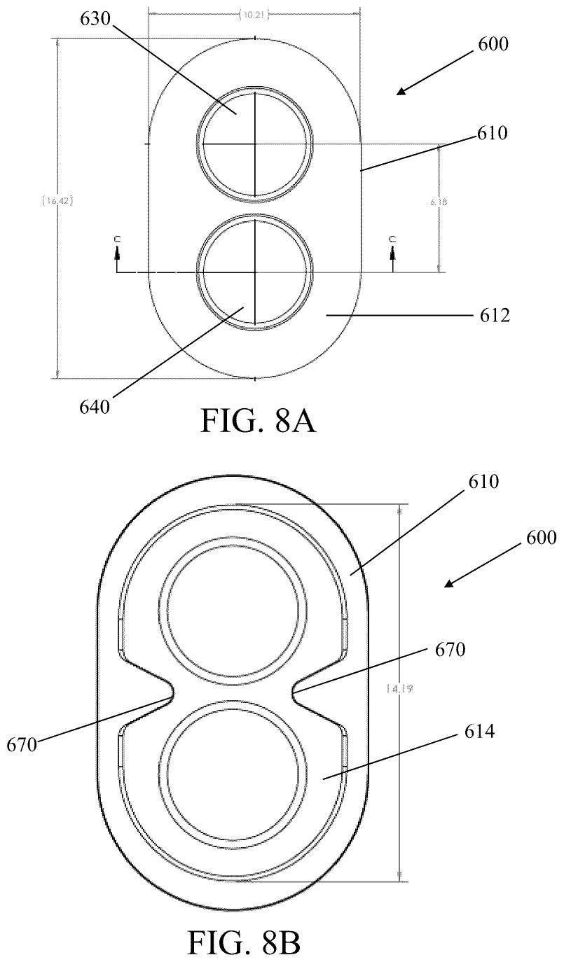

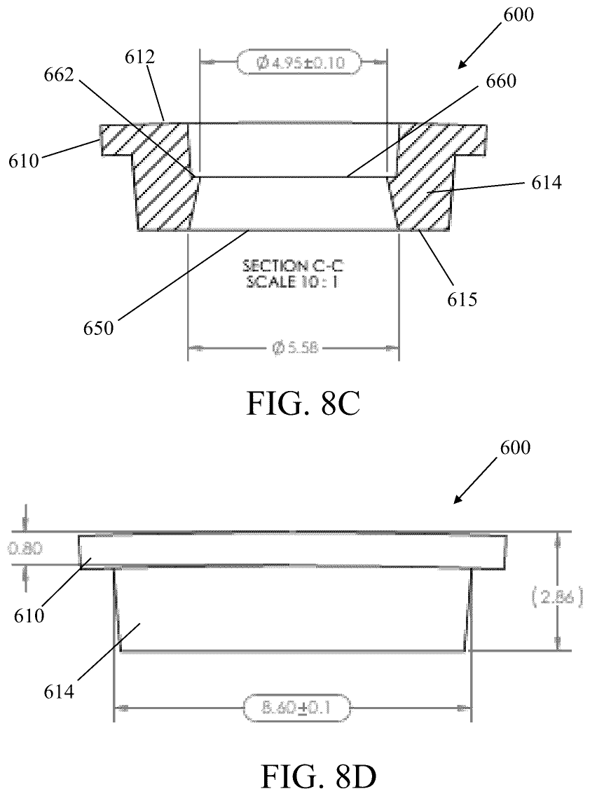

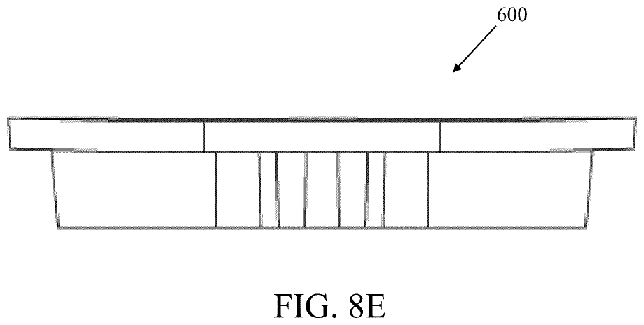

FIGS. 8A-8E depict various non-limiting views of the modified port insert 600. FIG. 8A is a top view of the modified port insert 600. FIG. 8B is a bottom view of the modified port insert 600. FIG. 8C is a cross-sectional, side view of the modified port insert 600. FIG. 8D is a side view of the modified port insert 600. FIG. 8E is a front, perspective view of the modified port insert 600. The dimensions included on FIGS. 8A, 8C and 8D are only representative of one non-limiting embodiment, and it will be appreciated that the components of the modified port insert 600 can have other dimensions.

With reference to FIGS. 8A-8E, and in accordance with the present non-limiting embodiment, the modified port insert 600 includes a top portion 610 having a top surface 612 and a bottom portion 614 that includes a bottom surface 615, and wherein the bottom portion 614 extends from the top portion 612. In one non-limiting embodiment, the modified port insert 600 further includes holes 630, 640 that extend through the top portion 610 and the bottom portion 614. In one non-limiting embodiment, each of the holes 630, 640 include a first diameter portion 650 and a second diameter portion 660. A landing 662 forms the divide between the first diameter portion 650 and the second diameter portion 660. In one non-limiting configuration, the holes 630, 640 located in the top portion either a) have a constant diameter from the top of the holes 630, 640 to the landing 662, or b) have a taper that narrows the diameter of the holes 630, 640 from the top of the holes to the landing 662. In another non-limiting configuration, the diameter of the holes 630, 640 in the bottom portion 614 and that is located at the landing is less than the diameter of holes 630, 640 just above the landing 662. In another non-limiting configuration, the diameter of the holes 630, 640 in the bottom portion 614 from the landing 662 to the bottom of the bod 501 either a) have a constant diameter from the landing 662 to the bottom of the holes 630, 640, or b) have a taper that widens the diameter of the holes 630, 640 from the landing 662 to the bottom of the holes. As illustrated in FIG. 3, the landing 662 and the diameter differences of the holes above and below the landing 662 facilitates in securing the modified port insert 600 to bosses 504, 506. When the bosses 504, 506 are formed of an elastomeric material, the outer surface of the bosses 504, 506 deforms about the varying diameters and/or cross-sectional areas in the holes 630, 640, thereby causing the modified port insert 600 to be secured to the bosses 504, 506.

In another non-limiting embodiment, the modified port insert 600 further includes undercut region 670 formed in the second portion 620. The undercut region 670 can be used to engage and fit into a specially shaped cavity region 107 of the modified port cavity 106 so as to inhibit or prevent rotation or movement of the modified port insert 600 in the cavity region 107 of the modified port cavity 106. Generally, the undercut region 670 is positioned partially in the space between the protrusion rings 504, 506.

With further reference to FIGS. 8A-8E, in one non-limiting embodiment, the modified port insert 600 is formed from one or more polymer materials (e.g. acrylonitrile butadiene styrene), metal, ceramic, etc.

In another non-limiting embodiment, the first portion 610 of modified port insert 600 optionally has a length of at least 2 mm, and typically 2-25 mm (and all values and ranges therebetween), and in one non-limiting configuration is 6.42 mm. In another non-limiting embodiment, the bottom portion 614 optionally has a length of at least 1.5 mm, and typically 1.5-24 mm (and all values and ranges therebetween), and in one non-limiting configuration is 14.19 mm. In another non-limiting embodiment, the top portion 610 optionally has a width of at least 2 mm, and typically 2-25 mm (and all values and ranges therebetween), and in one non-limiting configuration is 10.21 mm. In another non-limiting embodiment, the bottom portion 614 optionally has a width of at least 1.5 mm, and typically 1.5-24 mm (and all values and ranges therebetween), and in one non-limiting configuration is 8.60 mm. In another non-limiting embodiment, the modified port insert 600 optionally has a height of at least 0.1 mm, and typically 0.1-8 mm (and all values and ranges therebetween), and in one non-limiting configuration is 2.86 mm. In another non-limiting embodiment, the top portion 610 optionally has a height of at least 0.1 mm, and typically 0.1-3 mm (and all values and ranges therebetween), and in one non-limiting configuration is 0.80 mm. In one non-limiting embodiment, the distance between the center-points of the holes is similar or the same as the distance between the center-points of protrusion rings 504, 506. The diameter of holes 630, 640 is selected to be positioned about protrusion rings 504, 506.

The length of the protrusion rings 504, 506, the length of the air-ports 422, 424 extending from the bottom of the housing, and/or the diameter or cross-sectional area of bosses 112, 114 are selected to prevent the bottom ends of the air-ports 422, 424 from penetrating into the bladder B or from fully penetrating into the bladder B and contacting an opposite wall of the bladder B.

The modified port insert 600 facilitates in a) inhibiting or preventing damage to the ends of air ports 422, 424 during the connection of the housing to the cuff, b) inhibiting or preventing damage to the bladder B from the ends of air ports 422, 424 during the connection of the housing to the cuff, c) inhibiting or preventing damage to the ends of air-ports 422, 424 form contacting the bladder B during the connection of the housing to the cuff, thus inhibiting or preventing compression of the bladder B, which compression of the bladder B can interfere with the initial inflation of the bladder and/or the obtaining of correct pressure reading from the bladder during the inflation process, and/or d) maintains the separation of the bottom surface of the housing and the top surface of the modified port cavity 106. As illustrated in FIG. 3, the modified port insert 600 can be used to maintain a gap between the bottom surface of the housing component 124 of the air delivery unit 123 and the top surface of the modified port cavity 106 when the housing component 124 of the air delivery unit 123 is connected to the cuff 104 at least in the region of the modified port insert 600.

Referring again to FIG. 3, modified port cavity 106 can be connected to the top surface of cuff 104 so that a gap is formed between a portion of the bottom surface of the modified port cavity 106 and the cuff 104. Such gap allows a portion of the bladder B, during the inflation of the bladder B, to move upwardly into the gap to facilitate in the inflation of the cuff. As stated above, the ribs 512, 514, when located inside the bladder B and located at or near the one or more bosses, are configured to form spacing inside the bladder B to also facilitate in the inflation of the cuff in at least the region that air is inserted into the bladder B. The gap is formed between a portion of the bottom surface of the modified port cavity 106 and the cuff 104 further inhibits or prevents the compression together of the sides of the bladder B at the location that air is inserted into the bladder B, thus facilitating the proper inflation of the bladder B. As also discussed above, modified port insert 600 can be used to a) inhibit or prevent damage to the air ports 422, 424 of housing component 124 when the housing component 124 of the air delivery unit 123 is connected to the cuff 104, b) inhibit or prevent damage to one or more components of the modified cuff port arrangement 500 (e.g., body, bosses, protrusion rings, ribs) by the air ports 422, 424 of housing component 124 when the housing component 124 of the air delivery unit 123 is connected to the cuff 104, c) limit the distance that the air ports 422, 424 are inserted into the protrusion rings 504, 506 of modified cuff port arrangement 500 when the housing component 124 of the air delivery unit 123 is connected to the cuff 104, d) inhibit or prevent damage to the bladder B by the air ports 422, 424 of housing component 124 when the housing component 124 of the air delivery unit 123 is connected to the cuff 104, e) maintain the position of the protrusion rings 504, 506 of modified cuff port arrangement 500 as the air ports 422, 424 are inserted into the protrusion rings 504, 506 of modified cuff port arrangement 500 when the housing component 124 of the air delivery unit 123 is connected to the cuff 104, and/or f) provide structural integrity and/or support to the protrusion rings 504, 506 of modified cuff port arrangement 500 as the air ports 422, 424 are inserted into the protrusion rings 504, 506 of modified cuff port arrangement 500 when the housing component 124 of the air delivery unit 123 is connected to the cuff 104.

In operation, the modified port insert 600 is pressed into place such that the protrusion rings 504, 506 of modified cuff port arrangement 500 are pushed through the respective holes 630, 640 of modified port insert 600. When the air ports 422, 424 of housing component 124 are slide into the openings in the protrusion rings 504, 506, air-ports 422, 424 radially squeeze into the openings in the protrusion rings 504, 506 and the modified port insert 600 on the outside of the protrusion rings 504, 506. Because the modified cuff port arrangement 500 is welded to the bladder in the cuff 104, once the housing component 124 has been slid into the modified cuff port arrangement 500, it becomes difficult to separate the housing component 124 from the cuff 104.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall there between. The disclosure has been described with reference to the certain embodiments. These and other modifications of the disclosure will be obvious from the disclosure herein, whereby the foregoing descriptive matter is to be interpreted merely as illustrative of the disclosure and not as a limitation. It is intended to include all such modifications and alterations insofar as they come within the scope of the appended claims.

To aid the Patent Office and any readers of this application and any resulting patent in interpreting the claims appended hereto, Applicant does not intend any of the appended claims or claim elements to invoke 35 U.S.C. 112(f) unless the words “means for” or “step for” are explicitly used in the particular claim.

What is claimed:

1. A cuff system comprising an inflatable cuff that is configured to be releasably connected to a limb of a user, an inflation device that is configured to inflate said inflation cuff, and a cuff connection arrangement that is configured to connect said inflation device to said inflation cuff; said inflatable cuff includes an inflatable bladder; said inflation device includes a housing, one or more air-ports, and two or more of a) one or more motors, b) one or more pumps, c) power source, d) one or more pressure sensors and/or one or more pulse sensors, e) one or more circuit boards and other electronics, f) one or more processors, g) one or more pressure gauges, h) software, i) one or more displays, j) power/data transfer connector, k) air delivery unit, l) GPS system, m) wireless electronics, n) one or more press or contact buttons, o) one or more selection or scroll buttons, p) one or more switch(es), q) connection arrangement to permanently or releasably connect the housing to the cuff, r) one or more air-ports, s) one or more valves, t) one or more air tubes, u) one or more temperature sensors, v) gyroscope, w) one or more lights and/or other indicators to indicate a mode and/or operation and/or status, x) gyroscope, y) clock and/or z) timer; said cuff connection arrangement includes a modified port cavity and a modified cuff port arrangement; said modified port cavity is connected to said inflation cuff; said modified port cavity includes one or more connectors that are configured to connected to said housing; said modified cuff port arrangement is connected to said inflation cuff; said modified cuff port arrangement includes a body and one or more ribs, one or more bosses, and/or one or more protrusion rings; said one or more ribs are positioned on a top surface of said cuff and/or are located in an interior of said inflatable bladder; said one or more protrusion rings each include an ring opening that is adapted to receive at least a portion of one of said air-ports when said inflation device is connected to said cuff; said one or more bosses each form an opening through said body of said modified cuff port arrangement.

2. The cuff system as defined in claim 1, wherein said inflation device includes two of said air-ports and said cuff connection arrangement includes two of said protrusion rings wherein said ring opening in each of said protrusion rings is configured to telescopically received at least a portion of one of said air-ports.

3. The cuff system as defined in claim 1, wherein said one or more protrusion rings are connected to or formed on said modified cuff port arrangement; at least a portion of said one or more protrusion rings extends above a top surface of said cuff; said one or more ribs are connected to or formed on said modified cuff port arrangement; said one or more ribs are positioned in an interior of said inflatable bladder.

4. The cuff system as defined in claim 1, wherein said one or more protrusion rings are connected to or formed on a top surface of said body of said modified cuff port arrangement; said one or more ribs are connected to or formed on a bottom surface of said body of said modified cuff port arrangement; said ring opening in each of said protrusion rings includes a passageway through said protrusion ring that is at least partially aligned with said opening formed by one of said bosses.

5. The cuff system as defined in claim 1, wherein said modified cuff port arrangement is formed of a single piece of material.

6. The cuff system as defined in claim 1, wherein one or more of said bosses a) terminates at a top surface and a bottom surface of said body, b) extends upwardly from the top surface of said body and into at least a portion of an opening in said protrusion ring that is partially or fully aligned with said boss, and terminates at the bottom surface of said body, c) extends upwardly from the top surface of said body and into at least a portion of an opening in said protrusion ring that is partially or fully aligned with said boss, and extends downwardly from the bottom surface of said body, or d) terminates at the top surface of said body and extends downwardly from the bottom surface of said body.

7. The cuff system as defined in claim 1, wherein one or more of said ribs is positioned adjacent to or closely adjacent to one or more of said bosses.

8. The cuff system as defined in claim 1, wherein said modified port cavity is formed of a different material than said modified cuff port arrangement.

9. The cuff system as defined in claim 1, wherein said one or more protrusion rings extend upwardly from a top surface of said modified port cavity.

10. The cuff system as defined in claim 1, further including a modified port insert; said modified port insert is configured to be positioned above a top surface of said cuff and positioned at least partially about a portion of said one or more protrusion rings that extend upwardly from a top surface of said modified port cavity.

11. The cuff system as defined in claim 10, wherein said modified port cavity includes a recessed cavity region; a bottom portion of said modified port insert is configured to be positioned in said recessed cavity portion.

12. The cuff system as defined in claim 10, wherein said modified port cavity includes two port cavity openings; a portion of one of said protrusion rings is configured to be inserted through one of said port cavity openings.

13. The cuff system as defined in claim 12, wherein each of said port cavity openings in said modified port cavity has a variable cross-sectional area along a longitudinal length of each of said port cavity openings; each of said port cavity openings in said modified port cavity includes a landing.

14. The cuff system as defined in claim 1, wherein a bottom surface of said modified port cavity is spaced from a top surface of said cuff to form a gap region at or near said one or more protrusion rings.

15. An inflatable cuff that is adapted for use with an inflation device to inflate said inflatable cuff; said inflatable cuff includes an inflatable bladder and a cuff connection arrangement; said cuff connection arrangement is adapted to connect the inflation device to said cuff; said cuff connection arrangement includes a modified port cavity and a modified cuff port arrangement; said modified port cavity is connected to said inflation cuff; said modified port cavity includes one or more connectors that are configured to connected to the inflation device; said modified cuff port arrangement is connected to said inflation cuff; said modified cuff port arrangement includes a body and one or more ribs, one or more bosses, and/or one or more protrusion rings; said one or more ribs are positioned on a top surface of said cuff and/or are located in an interior of said inflatable bladder; said one or more protrusion rings each include an ring opening that is adapted to receive at least a portion of an air-port on the inflation device when the inflation device is connected to said cuff; said one or more bosses each form an opening through said body of said modified cuff port arrangement.

16. The inflatable cuff as defined in claim 15, wherein said cuff connection arrangement includes two of said protrusion rings; said ring opening in each of said protrusion rings is configured to telescopically received at least a portion of one of the air-ports when the inflation device is connected to said cuff.

17. The inflatable cuff as defined in claim 15, wherein said one or more protrusion rings are connected to or formed on said modified cuff port arrangement; at least a portion of said one or more protrusion rings extends above a top surface of said cuff; said one or more ribs are connected to or formed on said modified cuff port arrangement; said one or more ribs are positioned in an interior of said inflatable bladder.

18. The inflatable cuff as defined in claim 15, wherein said one or more protrusion rings are connected to or formed on a top surface of said body of said modified cuff port arrangement; said one or more ribs are connected to or formed on a bottom surface of said body of said modified cuff port arrangement; said ring opening in each of said protrusion rings includes a passageway through said protrusion ring that is at least partially aligned with said opening formed by one of said bosses.

19. The inflatable cuff as defined in claim 15, wherein said modified cuff port arrangement is formed of a single piece of material.

20. The inflatable cuff as defined in claim 15, wherein one or more of said bosses a) terminates at a top surface and a bottom surface of said body, b) extends upwardly from the top surface of said body and into at least a portion of an opening in said protrusion ring that is partially or fully aligned with said boss, and terminates at the bottom surface of said body, c) extends upwardly from the top surface of said body and into at least a portion of an opening in said protrusion ring that is partially or fully aligned with said boss, and extends downwardly from the bottom surface of said body, or d) terminates at the top surface of said body and extends downwardly from the bottom surface of said body.

21. The inflatable cuff as defined in claim 15, wherein one or more of said ribs is positioned adjacent to or closely adjacent to one or more of said bosses.

22. The inflatable cuff as defined in claim 15, wherein said modified port cavity is formed of a different material than said modified cuff port arrangement.

23. The inflatable cuff as defined in claim 15, wherein said one or more protrusion rings extend upwardly from a top surface of said modified port cavity.

24. The inflatable cuff as defined in claim 15, further including a modified port insert; said modified port insert is configured to be positioned above a top surface of said inflatable bladder and positioned at least partially about a portion of said one or more protrusion rings that extend upwardly from a top surface of said modified port cavity.

25. The inflatable cuff as defined in claim 24, wherein said modified port cavity includes a recessed cavity region; a bottom portion of said modified port insert is configured to be positioned in said recessed cavity portion.

26. The inflatable cuff as defined in claim 24, wherein said modified port cavity includes two port cavity openings; a portion of one of said protrusion rings is configured to be inserted through one of said port cavity openings.

27. The inflatable cuff as defined in claim 26, wherein each of said port cavity openings in said modified port cavity has a variable cross-sectional area along a longitudinal length of each of said port cavity openings; each of said port cavity openings in said modified port cavity includes a landing.

28. The inflatable cuff as defined in claim 15, wherein a bottom surface of said modified port cavity is spaced from a top surface of said cuff to form a gap region at or near said one or more protrusion rings.
